(19) United States
(12) Patent Application Publication
(10) Pub. No.: US 2009/0287209 A1
(43) Pub. Date: Nov. 19, 2009

(54) ABRASION CATHETER

(75) Inventors: Kazuo MATSUMOTO, Saitama (JP); Tetsui YAMASHITA, Hiroshima (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: JMS CO., LTD., Hiroshima (JP)

(21) Appl. No.: 12/121,200

(22) Filed: May 15, 2008

(51) Int. Cl. A61B 18/18

(52) U.S. Cl. 606/41

(57) ABSTRACT

An ablation catheter which has an electrode that can electrically cauterize a living body tissue at the tip side of a catheter, and this electrode is characterized in that the vibration and/or rotation of the electrode is controllable according to the temperature of the cauterization portion.

### FIELD OF THE INVENTION

The present invention relates to an ablation catheter, and, more especially to the ablation catheter used for an arrhythmic therapy.

### DESCRIPTION OF THE RELATED ART

An arrhythmia, which is caused by existence of the portions that has an excessive re-entry circuit and abnormal automaticity, can be controlled with the medicine of the kind of an anti-arrhythmic drug to control the abnormal electric activity. However, because the medical treatment by the anti-arrhythmic agent does not remove the abnormal area, it is not a treatment that cures abnormal heart rhythm fundamentally. Therefore, the ablation-therapy is performed instead of the medical treatment method that uses the medicine. The ablation-therapy is a treatment that burns off the living body tissue of the abnormal area.

The ablation therapy is performed as follows:

The ablation catheter specially designed for the purpose is used. In many cases, the ablation catheter is inserted into the body from the thick blood vessel (a femoral vein or a femoral artery) at the root of a leg, and an electrocardiogram is created by contacting the electrode to the inner wall of a heart. Based on the created electrocardiogram, a mapping work to find whether there is an abnormal area (For example, the portion leading to a tachycardia) is performed. When existence of an abnormal area is found by the mapping work, this abnormal area is cauterized by the high frequency from the electrode.

However, it is difficult for the ablation catheter currently used to control the area to be cauterized or the depth of cauterization in the abnormal area. In order to effectively control the factors, not only the output (energy) of ablator (high frequency generating equipment) is adjusted, but also the tip electrode of a catheter is actively cooled. For example, there are cooling methods for the electrodes as follows:

1. A method of cooling the electrode with the physiological saline solution spouted from the hole formed at the tip of an electrode like a sprinkler (a method using a irrigation catheter).
2. A method of cooling the electrode with the water that circulates through the inside of the circulatory circuit formed in the catheter (a method using a cooled chip).

By adopting these cooling methods, it is possible to decrease the danger of carbonization of the tissue, vaporization or formation of blood clot during the cauterization.

### Bibliography

### JP2005-518864

However, in these cooling methods, the structure and the system are complicated. Moreover, according to these cooling methods, there is also a risk of temperature exceeding 100° C. suddenly in the depths of the cardiac muscle, starting a steam eruption (it usually being called a pop phenomenon), and causing a phenomenon like the cardiac tamponaze by a explosion of the tissue. Furthermore, when cauterization using a high frequency ablation catheter is performed, the sudden rise of temperature in a local living body tissue causes the carbonization of the tissue, which results in a problem that the range of a cauterization is limited to small area, and cauterization depth also tend to be shallow because of the carbonization.

### BRIEF SUMMARY OF THE INVENTION

In order to solve the problem, the present invention provides the ablation catheter which has an electrode portion which is characterized by an ablation portion which is capable of vibrating and/or rotating.

### BRIEF DESCRIPTION OF THE DRAWING

FIG. 1 shows an embodiment of the ablation catheter of the present invention wherein a tip electrode can rotate. In FIG. 1, reference numeral 1 shows an ablation catheter, 2 shows the proximal electrode of an ablation catheter, 3 shows the tip electrode of an ablation catheter, and 4 shows an cauterization portion.

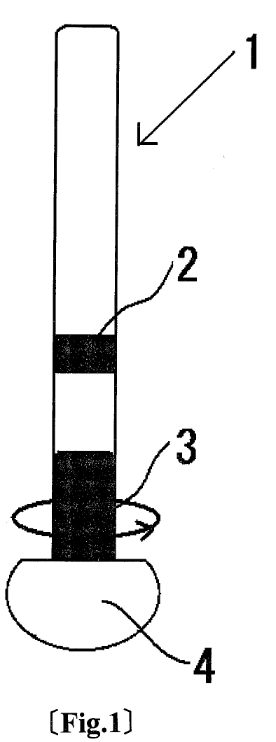

### DETAILED DESCRIPTION OF THE INVENTION

The ablation catheter of the present invention is characterized by an ablation electrode capable of vibrate and/or rotate which is formed at the tip of this catheter. During cauterization, the tip part of the ablation catheter can be cooled by a newly supplied blood flow that passes through between the portion currently cauterized and above-mentioned electrode part because of the vibration and/or rotation of the ablation electrode. Because the newly supplied blood flow is not heated, the electrode is cooled effectively.

The electrode of the conventional ablation catheter is also cooled by the blood flow except the part in contact with a cauterization portion. However, because the electrode of the conventional ablation catheter does not vibrate and/or rotate, this electrode contacts the heated cauterization portion. Therefore, the cooling effect of the electrode by the blood flow is low. On the other hand, the electrode of the ablation catheter of the present invention can draw the blood flow that is not heated into the space between the electrode tip part and the portion currently cauterized because of the vibration and/or rotation of the electrode. Therefore, the ablation catheter of the present invention can cool an electrode effectively as mentioned above, and can prevent formation of a blood clot, etc. Moreover, unlike the conventional cooling system, even if the ablation catheter of the present invention does not have a cooling means, it becomes possible to decrease the danger of carbonization of the tissue, vaporization, and pop phenomenon. Moreover, in addition to the function of the electrode, the ablation catheter of the present invention may have a cooling means like the conventional ablation catheter.

### DESCRIPTION OF PREFERRED EMBODIMENT

The electrode of the ablation catheter preferably can control the pitch of the electrode and/or the frequency of rotation of the electrode. For example, control of this electrode is performed based on the temperature detected by means to detect the temperature of the portions that is currently cauterizing. It is especially desirable to automatically control the electrode according to the temperature of the portion currently cauterized, the size of desired cauterization area, desired depth of the ablation, etc. In addition to an above-mentioned control of electrode, the electrode can also be controlled in consideration of the width of desired cauterization, and the amplitude of the vibration of an electrode is controlled in that case. The frequency of vibration of the electrode can be 5 Hz-1 kHz, for example. Moreover, the oscillating direction and rotating direction of the electrode may be arbitrarily chosen. For example, they can be in a transverse direction and/or a lengthwise direction.

Furthermore, the electrode of an ablation catheter can be controlled based on the cauterization result checked by the eyes of an operating person such as a doctor.

The control of the electrode can be not only by changing the frequency of vibration and/or number of rotations but also can be by turning ON and OFF depending on a cauterized state. For example, the ablation treatment is started in the OFF state which does not adopt vibration of the electrode, and if it is judged that this electrode needs to be controlled cooling, the electrode portion will be cooled by changing vibration of this electrode into ON state. Also, based on this cooling result, the frequency of vibration of the electrode can be controlled.

As a means to perform vibration of the electrode and/or rotation of an electrode, any means of vibrating and/or rotating can be chosen as long as the means can properly vibrate the electrode and/or rotate the electrode. The examples are an ultrasonic generating means, a micro motor, etc.

### EFFECT OF THE INVENTION

The ablation catheter of the present invention is simple in the structure or the system. Moreover, the ablation catheter of the present invention does not cause carbonization of the tissue by a sudden rise in a local temperature of a living body tissue or cardiac tamponaze. Furthermore, the ablation catheter of the present invention can easily control the range of cauterization and the depth of cauterization.

1. An ablation catheter comprising:
a catheter body, and
an electrode is capable of cauterizing a living body tissue electrically disposed at tip side of the catheter body, and
a vibrator and/or rotator to vibrate and/or rotate the electrode.

2. The ablation catheter according to claim 1, further comprising a controller to control frequency of vibration and/or number of rotations of the electrode.

3. The ablation catheter according to claim 2, further comprising a temperature detector to detect the temperature of an cauterization portion, wherein the frequency of vibration and/or the number of rotations of the electrode is controlled based on the temperature detected by the temperature detector.

4. The ablation catheter according to claim 2, wherein the controller not only controls the frequency of vibration and/or the number of rotations of the electrode, but also controls an amplitude of the vibration of the electrode.

5. The ablation catheter according to claim 2, wherein the controller controls the frequency of vibration and/or the number of rotations of the electrode based on a currently cauterized state of a cauterization portion.

6. The ablation catheter according to claim 2, characterized in that the the frequency of vibration of the electrode is 5 Hz-1 kHz.
